(19) United States
(12) Patent Application Publication
(10) Pub. No.: US 2012/0209081 A1
(43) Pub. Date: Aug. 16, 2012

(54) METHOD OF PREVENTING PATIENT INJURY

(76) Inventor: Abbas Sadeghian, Canton, OH (US)

(21) Appl. No.: 13/370,807

(22) Filed: Feb. 10, 2012

Related U.S. Application Data
(60) Provisional application No. 61/441,872, filed on Feb. 11, 2011.

(51) Int. Cl. A61B 5/00

(52) U.S. Cl. 600/300

(57) ABSTRACT

This invention relates to administering a test to an incoming patient to determine that patient's ability to delay gratification and resist impulses, and thereafter grouping the patient into a high risk or low risk group. The patients in the high risk group are thereafter attended to at a greater frequency as it has been determined that the patient cannot effectively delay gratification and resist impulses. The preferred embodiment of this invention uses the Stroop Neuropsychological Screening Test to identify which patients cannot effectively delay gratification. Patients in the high risk group may be toileted more frequently to eliminate the impulse to move around the facility to remove bladder or bowel pressure.

### CROSS-REFERENCE TO RELATED APPLICATION

This application claims priority from U.S. Provisional Application Ser. No. 61/441,872, filed Feb. 11, 2011, the disclosure of which is incorporated herein by reference.

### BACKGROUND OF THE INVENTION

1. Technical Field

This invention relates to patient management within a healthcare facility. More particularly, this invention relates to preventing patient injury by reducing the number of patient falls within the facility. Specifically, this invention relates to administering a test to an incoming patient to determine that patient's ability to delay gratification or resist impulses, and thereafter toileting or otherwise attending to the patient at a greater frequency if it is determined that the patient cannot effectively delay gratification or resist impulses.

2. Background Information

Patients who accidentally fall during a stay in a healthcare facility constitute a major cause of complicating injuries, lawsuits, and even death. Families are distressed, and healthcare providers feel anxiety and guilt. Finding a more effective way of predicting which patients are most likely to behave in a way that will lead to a fall can prevent injury and the cascade of negative consequences that ensue.

While any patient could potentially fall in a healthcare setting, older patients are particularly at risk for accidental falls. According to recent studies, approximately thirty percent of people over sixty-five years of age fall each year, and the number is higher among those living in institutions. Six percent of all people over sixty-five require medical attention for a fall. Among that elderly group, injury from a fall often results in high morbidity, mortality, and a need for additional healthcare services, including premature nursing home admissions. Families of patients often wonder how injuries can happen in a healthcare setting, which they assume is a place of safety. That can lead to feelings of anger and the need to blame someone. Accordingly, the treating staff feels anxiety and guilt when a patient is injured in their care.

For all these reasons, healthcare providers have increasingly focused on risk management and safety devices to prevent accidental falls. For example, patients are typically issued socks with rubber treading on the soles to provide greater friction between the sock and the floor while the patient is walking around the facility. However, these accidents are not an inevitable result of treating the elderly. Studies have identified a diverse group of risk factors for falls of different types in a variety of settings and patient groups. These have helped identify high-risk groups and resulted in a range of testing instruments that can further focus on identifying specific at-risk patients. As such, multi-factorial assessment tools have been developed to assess the patient and to try to predict those at a higher risk of falling. However, these multi-factorial assessment tools are generally only accurate in determining which patients among an already “at-risk” group such as the elderly, are most likely to fall. Furthermore, these multi-factorial tests are lengthy and time-consuming and typically require a professional psychologist or medical doctor to administer the test. This drives up hospital costs and dramatically increases the time it requires to properly receive a patient into the facility. Further, these test do not account for any medicines the patient may be taking during their stay at the healthcare facility and the resulting affect on the patient's behavior.

Prevention of falls and the result injury in healthcare facilities is critical. As such, facilities often are overly aggressive in grouping patients into a high-risk category in an attempt to “cast a wide net” and prevent false negatives. However, in doing so, many false positives are created and many patients who are considered high-risk are not, and therefore do not require the additional hospital resources to prevent a fall.

Millions of dollars in insurance premiums and lawsuit judgments are spent each year by healthcare facilities throughout the world due to patient falls. Therefore, there is a great need in the art to provide a method for reducing patient falls within a healthcare facility. This method should include a procedure for non-psychologists and non-doctors on the facility staff to quickly identify which patients are at a high risk of falls. The procedure should dramatically reduce false negatives and false positives. Furthermore, the method for reducing patient falls should also include a procedure to prevent patient falls once the high risk group has been identified.

### BRIEF SUMMARY OF THE INVENTION

This invention relates to administering a test to an incoming patient to determine that patient's ability to delay gratification or resist impulses, and thereafter toileting or attending to the patient at a greater frequency if it is determined that the patient cannot effectively delay gratification or resist impulses. The preferred embodiment of this invention uses the Stroop Neuropsychological Screening Test to identify which patients cannot effectively delay gratification or resist impulses. Thereafter, in the preferred embodiment, those patients who are found to be unable to delay gratification or resist impulses are attending to with a greater frequency, preferably by toileting the patient approximately once every one to two hours to eliminate the impulse to move around the facility to remove bladder or bowel pressure.

### BRIEF DESCRIPTION OF THE SEVERAL VIEWS OF THE DRAWINGS

A preferred embodiment of the invention, illustrated of the best mode in which Applicant contemplates applying the principles, is set forth in the following description and is shown in the drawings and is particularly and distinctly pointed out and set forth in the appended claims.

FIG. 1 is a flow-chart depicting the steps in a first embodiment of the present invention for preventing patient injury in a healthcare facility; and

FIG. 2 is a flow-chart depicting the steps in a second embodiment of the present invention for preventing patient injury in a healthcare facility.

Similar numbers refer to similar parts throughout the drawings.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

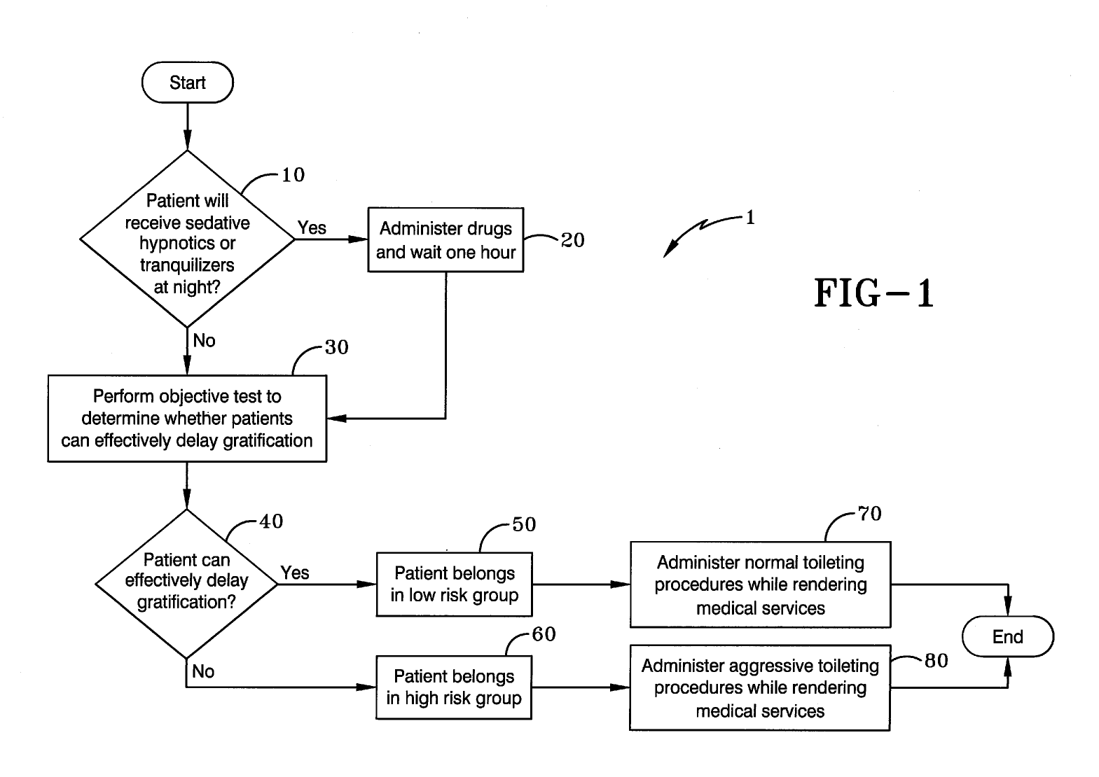

Referring now to FIG. 1, there is shown a method 1 for reducing patient falls within a healthcare facility. Method 1 has been proven to consistently determine which patients are at a high risk of falling, and thereafter successfully prevent the patients from falling without resorting to immobilizing the patient. At the outset, it must be understood that a unique discovery has been made as to the correlation of gratification delay with patients falling in a healthcare facility. Further, a method of preventing the falls has also been discovered once the group of high risk patients has been identified.

Deferred gratification and delayed gratification denote a person's ability to wait in order to obtain something that he or she wants. This intellectual attribute is also called impulse control, will power, self control, and “low” time preference, in economics. Moreover, people who lack the psychological trait of being able to delay gratification are said to require instant gratification and might suffer poor impulse control. In a healthcare setting, patients may lack adequate impulse control or due to a variety of factors, including illness, age, brain trauma, or medication. The phrase “effectively delay gratification” is used herein to describe a person's ability to wait to obtain something he or she wants, and further denotes the ability to exercise typical impulse control for an average human adult.

One of the most basic human impulses is the need to relieve oneself of bladder or bowel pressure from bodily waste buildup. Relieving this pressure provides immediate gratification to the person at a very primitive level, as this human impulse is present since before birth. Thus, it has been discovered that patients who cannot effectively delay gratification, cannot resist the urge to physically move to a bathroom if at all possible to relieve the bladder or bowel pressure. Oftentimes this inability to delay the gratification of relieving waste pressure results in patients moving about the facility as soon as they feel this pressure or sensation, which results in patients moving about the facility unassisted and unsupervised. The patients may even appropriately actuate the nurse “call button,” but when help doesn't immediately arrive, the patients with poor impulse control cannot resist the impulse to look for a bathroom. As these patients are typically unfamiliar with the facility and unassisted, falls often result. It has been discovered that the concepts of gratification delay and falling directly correlate. As such, identifying which patients cannot effectively delay gratification also identifies which patients are at a high-risk of falling. It has further been discovered that the reverse is also true. Identifying which patients can effectively delay gratification also identifies which patients are at a low-risk of falling.

Based on this discovery, a procedure for preventing patient falls once the high risk group has been identified has also been realized. Inasmuch as the impulse to ambulate to a bathroom is typically the most common and most frequent impulse felt by a patient, it has been discovered that by simply reducing the possibility that these patients will feel bladder or bowel pressure, these patients will not be stimulated to move about the facility. By increasing the frequency with which the facility staff toilets these high-risk patients, bladder or bowel pressure is prevented. As a direct result of removing this frequent impulse to ambulate to a bathroom, unassisted patient movement and subsequent falls are thereby eliminated.

As shown in FIG. 1, according to an initial step 10, the patient is interviewed and accessed to determine if the patient will receive hypnotic sedatives or tranquilizers during their stay at the healthcare facility. These substances have been shown to affect the mental functions of the patient, and specifically the areas of the brain dealing with gratification delay. Thus, it has been found that these patients must be evaluated with respect to their ability to delay gratification while under the influence of these types of drugs. If it is determined in step 10 that the patient will not receive these drugs while at the healthcare facility, method 1 proceeds to a step 30. Alternatively, if it is determined in step 10 that the patient will receive hypnotic sedative or tranquilizers during their stay at the healthcare facility, method 1 proceeds to a step 20. While step 10 describes hypnotic sedatives or tranqulizers in particular, if the patient is to receive any drugs, or is currently on any proscribed medications, the present invention encompasses administering these drugs prior to the following steps. Therefore, administering any medications prior to the following steps is within the scope of this invention.

According to step 20, the patient is administered the same hypnotic sedatives or tranquilizers, or any necessary drugs, which the patient will be receiving during the patient's stay at the healthcare facility. An approximately one hour wait is then instituted for the respective drugs to take effect to ensure that the patient will be under the influence of these drugs during step 30. This is a primary feature of the present invention and a novel concept within the art, as no previous fall screening method or fall risk-assessment test incorporates into the assessment the effect of a patient's particular required drugs on their assessment or test score. These drugs typically affect the same areas of the brain that control automatic behaviors, executive functions, and gratification delay, namely, the frontal lobe. Thus, they must be accounted for and incorporated into the screening method to provide an accurate picture of how that particular patient's brain will function during their stay at the healthcare facility. Once the patient is under the influence of these drugs, in approximately one hour after administration, method 1 can proceed to step 30. However, given that medications are biochemically delivered at differing rates, step 20 includes waiting an effective amount of time for the administered medications to take effect. A one hour wait is simply the exemplary time for step 20, waiting any effective amount of time to ensure the medications have taken effect is within the scope of this invention.

According to step 30, a member of the facility staff will perform a test on the patient to determine whether the patient can delay gratification or resist impulses. This test will typically produce a numbered result, which only must be compared to a threshold value to determine whether the patient belongs in the high-risk group the low-risk group. Thus, after properly administering a test which adequately measures whether the patient can delay gratification, there will be no subjectivity in interpreting the test results. For example, if the test result is greater than the threshold value, the patient belongs in the low-risk group, and if the test result is less than the threshold value, the patient belongs in the high-risk group. It will be readily understood that a test could be devised to provide where greater than the threshold value indicates high-risk, and below the threshold value indicates low risk. As such, a non-psychologist such as a nurse can administer the test and review the results, saving the healthcare facility time and money. Thus, a sub-doctorate level employee of the healthcare facility may administer the test. As used herein, “sub-doctorate level” employee is an employee which has not received a medical degree or a doctorate degree. This term is used to identify lower cost employees, from the perspective of the healthcare facility.

It is a primary feature of the invention that threshold values of when patients are considered “high-risk” are configurable by the healthcare facility itself, and even configurable down to the unit or type of ailment to which the patient is being admitted. For example, a healthcare facility may wish to lower the threshold value for when patients are considered high-risk if the patient is being admitted for a stroke, which typically affects the brain, and specifically the frontal lobe which helps to control physical movement. Conversely, the healthcare unit may wish to raise the threshold value for when patients are considered high-risk if the patient is being admitted into a burn unit or other injuries or ailments which typically do not affect patient mobility.

In the preferred embodiment of the present invention, the patient is administered the Stroop Neuropsychological Screening Test in step 30 to objectively determine whether the patient can delay gratification. This test is well known in the art, and as discussed previously provides a numbered test result to the administrator of the test. This test result indicates what percentile of the population the patient ranks in terms of cognitive flexibility, which includes the ability to delay gratification. Though representing a percentile, the test result is simply a number which can be compared to a given threshold value to determine if the patient is high-risk or low-risk. Thus, a non-psychologist and non-medical doctor can readily determine by considering the test result whether the patient can effectively delay gratification or cannot. Thus, lesser paid and lesser trained facility staff can administer the test and consider the result. This represents an enormous cost-savings to a typical healthcare facility.

After administration of the test in step 30, the test results are reviewed in a step 40 and a determination is made as to whether the patient can delay gratification or cannot. If it is determined that the patient can delay gratification, method 1 proceeds to a step 50, wherein the patient is assigned to a low risk group of patients. Conversely, if the patient cannot delay gratification, method 1 proceeds to a step 60, wherein the patient is assigned to a high risk group of patients. Thus, the test administered and reviewed in step 30 and step 40, respectively, quickly and directly determines the fall risk of the patient. Thus, the patient can be quickly assigned to a corresponding risk-based group. It is envisioned that step 10, step 20, step 30, step 40, step 50, and step 60 can be performed by non-psychologists and non-doctors, preferably by a nurse. It is also envisioned that these steps may be performed during the client intake process, as these steps represent a relatively small amount of time. Thus, it is a primary feature of the invention that step 10, step 20, step 30, step 40, step 50, and step 60 can be performed relatively quickly to allow the healthcare facility to assign the patient to the correct risk-based group during the initial intake of the patient.

Once the patient is assigned to the correct risk-based group, either in step 50 representing a low-risk group, or step 60 representing a high-risk group, the patient is administered the required medical services by the healthcare facility. However, if the patient is assigned to the low-risk group (step 50), medical services are administered in a step 70. During step 70, normal procedures for toileting the patient are observed. The typical frequency for toileting a patient which is considered to have a low risk of falling is administered. This is typically once every three to four hours in a typical hospital setting. While step 70 indicates normal toileting procedures will be undertaken, step 70 includes any common hospital procedures, and includes these procedures being done at the typical rate or frequency. Thus, step 70 encompasses not only toileting the patient at a normal rate, but may also include any hospital functions or procedures being attended to at a typical or normal rate as understood in the industry.

If the patient is assigned to the high-risk group (step 60), medical services are administered in a step 80. During step 80, aggressive toileting procedures are observed for the patients in the high-risk group. The typical frequency in which a patient is toileted is greatly increased in the hopes that the patient will never feel the sensation that the patient needs to relieve himself. It has been discovered that patients who cannot delay gratification correspondingly cannot delay the impulse to get out of bed, a chair, or a sitting position to go to the bathroom and relieve the unpleasant sensation of a full bladder or bowels. Thus, the gratification of relieving the bladder or bowl pressure cannot be effectively delayed by patients in the high-risk group. Without this higher order of cognitive function of gratification delay, the patient will move out of a generally safe position and try to go to the bathroom, thus putting that patient at a high risk of falling while undertaking this movement. Thus, it is a primary feature of the invention that the ability to delay gratification can be correlated with fall risk through the impulse driven act of relieving bladder or bowel pressure. It is another primary feature of this invention that the high-risk of the patient falling can thereby be neutralized by frequently toileting the patient such that the impulse to relieve bladder or bowel pressure is never realized by the patient. It has been found through experimentation that toileting patients generally in the range of every one to two hours is sufficient to prevent bathroom impulses in the patent. As such, toileting the patients in the high-risk group approximately twice as frequently as the patients in the low-risk group has also been found to be sufficient.

While step 80 indicates aggressive toileting procedures will be undertaken, step 80 includes any common hospital procedures, and includes these procedures being done at an aggressive rate or frequency. Thus, step 80 encompasses not only toileting the patient at an aggressive rate, but may also include any hospital functions or procedures being attended to at an aggressive frequency or aggressive rate as understood in the industry.

It is another primary feature of the present invention that method 1 prevents false positives as well as false negatives, allowing the healthcare facility to allocate resources much more efficiently. A false negative results in a patient who is a high-risk being placed under normal care by the healthcare facility. This is the situation every facility is trying to avoid, as this situation results to falls and injuries. However, the reluctance to “miss” a potential high-risk patient leads to many facilities being overly aggressive in placing any potential fall risks into the high-risk category, thus creating many false positives. This situation unnecessarily drains hospital resources because many patients who are not a fall risk are thereby placed under aggressive care by the healthcare facility. By accurately determining fall risk in step 30 and step 40, patients are assigned to the correct group and the proper toileting frequency is administered, thus allowing the healthcare facility to allocate resources much more efficiently.

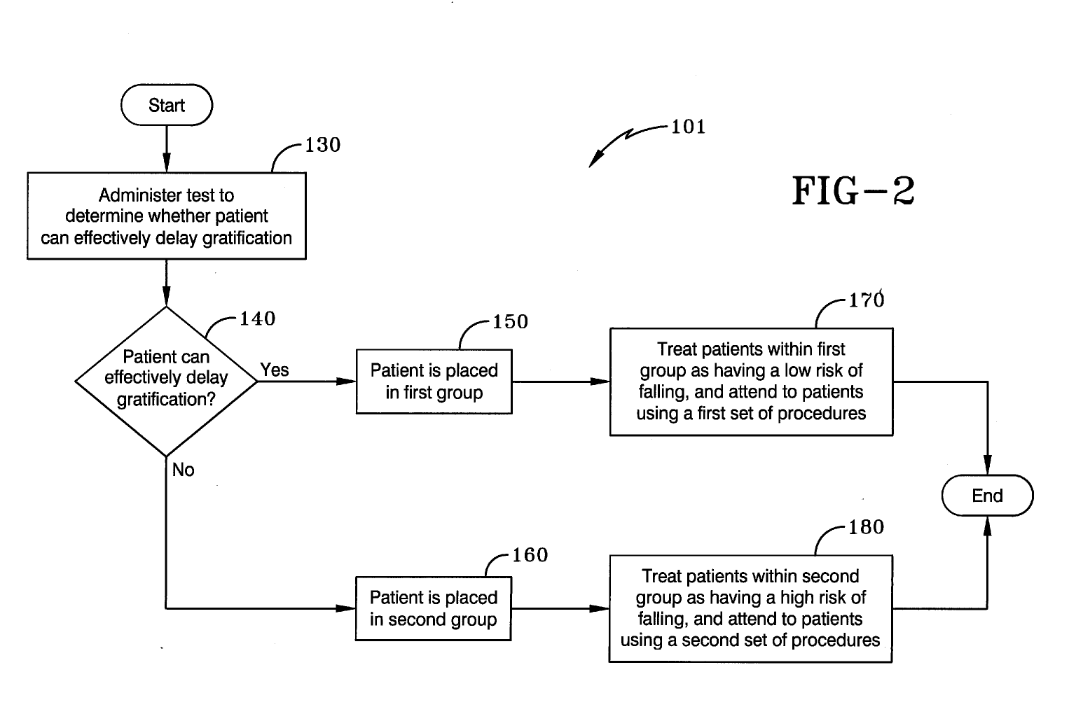

Referring now to FIG. 2, there is shown a method 101 for reducing patient falls within a healthcare facility. Method 101 is a more generalized approach to preventing patient falls, with respect to method 1. Method 101 is initiated by a step 130 where an incoming patient is administered a test to determine whether the patient can effectively delay gratification. After this test is administered, a step 140 is undertaken where the test administrator or another decision maker determines from the results of the testing in step 130 whether the patient can effectively delay gratification. If so, method 101 proceeds to a step 150, where the patient is placed in a first group. If the patient cannot effectively delay gratification, step 140 proceeds to a step 160, where the patient is placed in a second group. As more patients are admitted into the healthcare facility, they are similarly divided into either the first group or the second group. Step 150 proceeds to a step 170, where the patients are attended to by using a first set of procedures. Step 160 proceeds to a step 180, where the patients are attended to by using a second set of procedures. The first set of procedures differ from the second set of procedures in that the second set of procedures attend to the patient's needs in the second set at a higher frequency or at an aggressive rate, as this second group is considered to be at a higher risk of falls.

One task which may be included in both the first set of procedures and the second set of procedures is the task of toileting the patient. Thus, toileting the patients in the second set is done at a more aggressive rate or more frequently, when compared to rate at which the patients in the first group are toileted. Other similar tasks may be included in each of the first set of procedures and the second set of procedures.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A method adapted to treat a patient within a healthcare facility, the method comprising the steps of:
administering a test to the patient to determine whether the patent can effectively delay gratification;
grouping the patient into a high risk group of patients if it is determined that the patient cannot effectively delay gratification;
grouping the patient into a low risk group of patients if it is determined that the patient can effectively delay gratification; and
attending to the high risk group of patients more frequently than the low risk group of patients.

2. The method of claim 1, further comprising the step of toileting the high risk group of patients more frequently than the low risk group of patients.

3. The method of claim 1, wherein the test is the Stroop Neuropsychological Screening Test.

4. The method of claim 1, wherein the test is adapted to be administered by a sub-doctorate level employee of the healthcare facility.

5. The method of claim 1, further comprising the step of administering any currently proscribed medication to the patient and waiting for the medication to take effect prior to administering the test.

6. The method of claim 1, further comprising the step of administering at least one of a sedative hypnotic and a tranquilizer to the patient and waiting for the at least one of the sedative hypnotic and the tranquilizers to take effect prior to administering the test.

7. A method for reducing patient falls within a healthcare facility, the method comprising the steps of:
determining whether each incoming patient belongs in a low risk group of patients or a high risk group of patients; and
toileting the high risk group of patients more often than the low risk group of patients.

8. The method of claim 7, further comprising the step of determining whether each patient can effectively delay gratification, wherein patients who can effectively delay gratification are assigned to the low risk group of patients, and wherein patients who cannot effectively delay gratification are assigned to the high risk group of patients.

9. The method of claim 7, further comprising the steps of:
determining a threshold value for when a patient is considered to have a high risk of falling;
administering the Stroop Neuropsychological Screening Test to produce a test result; and
comparing the test result to the threshold value to determine whether the patient belongs in the low risk group of patients or the high risk group of patients.

10. The method of claim 9, wherein the threshold level is set at the 50^{th} percentile, such that a patient receiving a test result of 50 or greater on the Stroop Neuropsychological Screening Test are assigned to the high risk group of patients.

11. The method of claim 9, wherein the high risk group of patients are toileted every one to two hours.

12. The method of claim 9, wherein the test is adapted to be administered by a sub-doctorate level employee of the healthcare facility.

13. The method of claim 7, further comprising the steps of:
administering at least one of the patient's currently proscribed medicines;
waiting an effective amount of time for the at least one of the patient's currently proscribed medicines to take effect; and
determining whether the patient belongs in the high risk group of patients or the low risk group of patients while the patient is under the influence of the at least one of the patient's currently proscribed medicines.

14. The method of claim 13, further comprising the step of determining whether each patient can effectively delay gratification, wherein patients who can effectively delay gratification are assigned to the low risk group of patients, and wherein patients who cannot effectively delay gratification are assigned to the high risk group of patients.

15. The method of claim 14, wherein the patients are administered the Stroop Neuropsychological Screening Test to objectively determine whether the patients are able to delay gratification, wherein patients who pass the Stroop Neuropsychological Screening Test are assigned to the low risk group of patients, and wherein patients who do not pass the Stroop Neuropsychological Screening Test are assigned to the high risk group of patients.

16. The method of claim 14, wherein the high risk group of patients are toileted every one to two hours.

17. The method of claim 14, wherein the test is adapted to be administered by a sub-doctorate level employee of the healthcare facility.

18. A method of rendering medical services in a healthcare facility to a patient requiring one of sedative hypnotics and tranquilizers while in the healthcare facility, comprising the steps of:
administering said one of sedative hypnotics and tranquilizers;
waiting an effective amount of time for the said one of sedative hypnotics and tranquilizers to take effect;
administering a test to the patient to determine whether the patient belongs in a low risk group of patients or a high risk group of patients; and
attending to the patients in the high risk group of patients more frequently than the patients in the low risk group of patients.

19. The method of claim 18, wherein the test measures the ability to effectively delay gratification, wherein the patient is placed in the high risk group of patients if the test results indicate that the patient is not able to effectively delay gratification, and wherein the patient is placed in the low risk group of patients if the test results indicate that the patient is able to effectively delay gratification.

20. The method of claim 19, wherein the patients in the high risk group of patients are toileted more frequently than the patients in the low risk group of patients.
